(19) United States
(12) Patent Application Publication
(10) Pub. No.: US 2009/0036848 A1
(43) Pub. Date: Feb. 5, 2009

(54) PORTABLE URINAL AND METHOD FOR USE

(75) Inventor: Norman A. Faiola, Ithaca, NY (US)

Correspondence Address: WOOD, HERRON & EVANS, LLP, 2700 CAREW TOWER, 441 VINE STREET, CINCINNATI, OH, 45202, US

(73) Assignee: THE VERACITY GROUP, INC., Mainevill, OH (US)

(21) Appl. No.: 11/832,671

(22) Filed: Aug. 2, 2007

(51) Int. Cl. A61F 5/44; A47K 11/00

(52) U.S. Cl. 604/329; 4/144.1

(57) ABSTRACT

A portable urinal has a lid or projecting member from the housing with a mirror attached to allow the urinal user to properly aim his or her urine stream. The mirror helps users prevent embarrassing accidents and spills when the portable urinal is used in situations such as by bed-ridden patients in hospitals. The portable urinal is filled in a generally horizontal position but is designed to sit vertically on a table or be hung by a handle on a hospital bed rail. The mirror lid or projection can be permanently or removably coupled to the portable urinal to allow for easy urine disposal, cleaning, and sterilization.

### FIELD OF THE INVENTION

This invention relates to portable urinals and more particularly pertains to a portable urinal with a reflective device to assist the urinal user avoid spills and messes.

### BACKGROUND

Portable urinals are devices, usually hand-held, that are used to collect urine during urination. For example, a portable urinal called a bed urinal is necessary for a patient confined to a hospital bed. A bed urinal used by a patient is susceptible to spills and leaks both while the patient is using the urinal as well as when the urinal is moved by the patient after use. This happens because it is very unlikely that a patient lying in a bed can adequately see the urinal opening and aim a urine stream accordingly. This may be true for patients with neck injuries, vision problems or obesity issues. Such spills are not desirable because they can create a health risk and must be cleaned up immediately. Furthermore, the patient may feel embarrassed and uncomfortable if the spill is in the patient's bed or on the patient's body or clothing.

Most portable urinals are made in an economical manner by blow molding and have snap-on lids to cover the primary opening for urine. Some portable urinals in the prior art have tried to solve the spillage problem for when the patient is moving the portable urinal after use. Examples are shown in U.S. Pat. Nos. 5,797,147; 5,953,763; 6,021,529; and 6,588,024.

Nevertheless, improvements to facilitate ease of use and minimize spillage while the urinal is in use are highly desired.

### SUMMARY OF THE INVENTION

This invention in one embodiment relates to a portable urinal designed to maintain a user's dignity and minimize spills caused by poor aim during urination. The portable urinal includes a container with a neck portion and an opening at one end to accept urine into the container. Many of these portable urinals will also have an attached handle to be grasped and a lid to cover the opening. According to various embodiments of this invention, a mirror is attached to the lid or to a projection from the exterior of the container in the case where there is no lid on the container. This mirror can be moved around with the same hand holding the handle of the container until the user can see properly through the reflection where he is aiming his urine stream.

After urination, the user can then easily snap the lid back on the opening and move the container for storage or draining. In the case of the portable urinal being used for a hospital patient, the handle can be used to hang the filled container on a bed rail. The end of the container opposite the opening and neck portion may be flat so that the portable urinal can be stored sitting upright on a horizontal surface. Additional features of the invention will be described hereinafter and will form the subject matter of the claims below.

### BRIEF DESCRIPTION OF THE DRAWINGS

The accompanying drawings, which are incorporated in and constitute a part of this specification, illustrate exemplary embodiments of the invention and, together with a general description of the invention given above, and the detailed description given below, serve to explain the principles of the invention.

FIG. 1 is a perspective view of a portable urinal according to one embodiment of the invention with a lid member covering an opening.

FIG. 2 is a perspective view of the portable urinal in FIG. 1 with a mirror on an interior surface of the lid member being visible.

FIG. 3 is a perspective view of the lid member with a mirror and projecting member.

FIG. 4 is a perspective view of another embodiment of the invention where the lid member is permanently coupled to the handle member.

### DETAILED DESCRIPTION

A portable urinal 10 according to a first embodiment of this invention is illustrated in FIGS. 1-3. The urinal 10 has a housing 12 which defines a perimeter wall 14. The perimeter wall 14 defines an interior surface 16 of the housing 12. The interior surface 16 includes an end wall 18, one or more side walls 20, and an opening 22 opposite the end wall 18. The opening 22 allows urine to pass into the housing 12 for storage and disposal. The side walls 20 in the embodiment of FIGS. 1-3 define a rectangular cross-section, but other suitable side wall cross-sections may be used. The configuration of the urinal 10 shown in FIG. 1 is typically useful for male users and other configurations of the urinal 10 are adapted for female users within the scope of this invention.

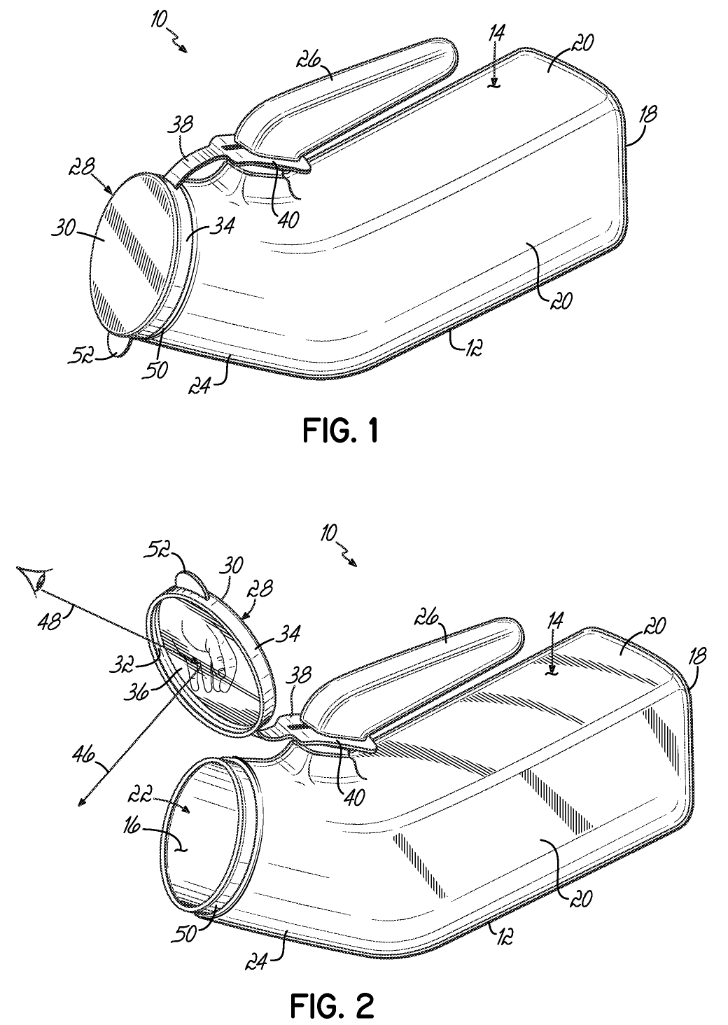

The side walls 20 are bent between the opening 22 and the end wall 18 to create a neck portion 24 where the opening 22 is located. This neck portion 24 stops urine from pouring out of the housing 12 when the housing 12 is held generally horizontal. The neck portion 24 in the embodiment shown is circular, but any suitable cross-section may be provided. As seen in FIGS. 1-2, a handle member 26 is coupled to the housing 12 close to the opening 22 to allow for easy handling and moving of the portable urinal 10.

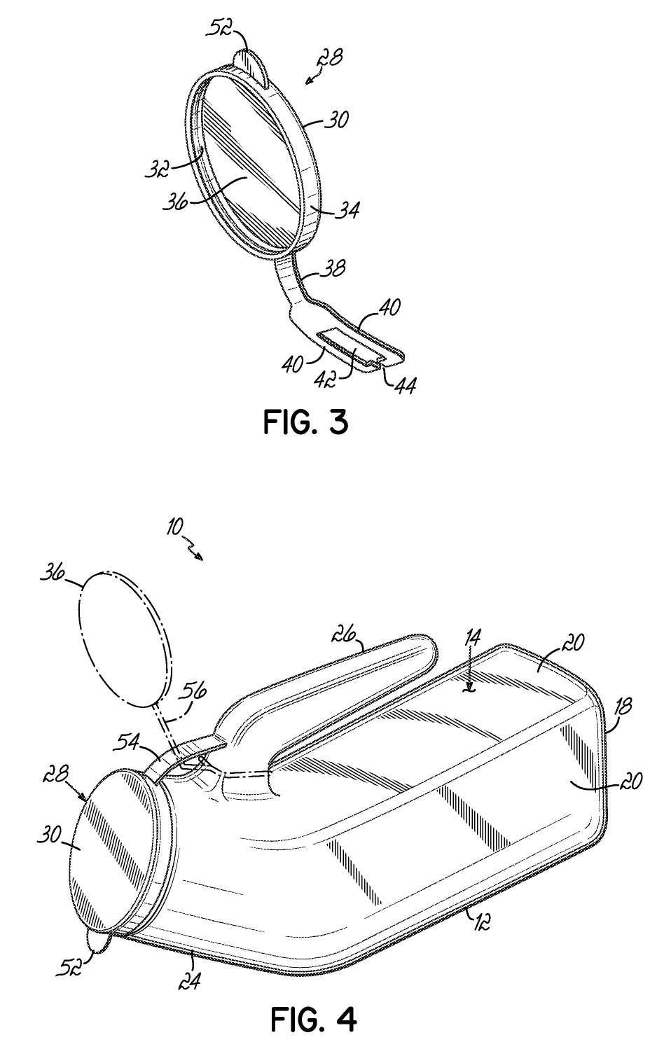

A lid member 28 used on the portable urinal 10 in FIGS. 1-2 is shown in greater detail in FIG. 3. The lid member 28 includes an exterior surface 30, an interior surface 32, and a shoulder 34 adapted to fit over the opening 22 at the end of the neck portion 24. The neck portion 24 may have a recessed surface 50 adjacent to the opening 22 to allow the shoulder 34 of the lid member 28 to easily fit over the opening 22. The interior surface 32 of the lid member 28 is covered by a mirror or other reflective surface 36 attached to the interior surface 32. The mirror 36 can be made of glass material, but for durability and safety reasons will probably be made of acrylic material in most embodiments. The shoulder 34 has a lid tab 52 projecting from one side which helps a user grab and remove the lid member 28 from the opening 22. The shoulder 34 also has a projecting member 38 extending from it to couple the lid member 28 to the portable urinal 10.

In the embodiment shown in FIGS. 1-3, the projecting member 38 includes two legs 40 which define an aperture 42 through the projecting member 38. The aperture 42 has an opening 44 between the two legs 40 so that the projecting member 38 can be clipped onto or coupled to the handle member 26 as shown in FIGS. 1-2. The handle member 26 can also be slid through the aperture 42 to couple the two components. Either manner of coupling the lid member 28 to the housing 12 will allow the lid member 28 to be pivoted proximate to the opening 22 and also makes the lid member 28 removable if necessary for cleaning or otherwise.

FIG. 1 shows the portable urinal 10 with lid member 28 covering the opening 22. A method for using the portable urinal 10 would begin with placing the portable urinal 10 generally horizontal as shown in FIGS. 1-2 between the legs of a person in a bed. The lid member 28 can then be removed from the opening 22 and pivoted upward so that the mirror 36 can show the reflection shown in FIG. 2. The mirror 36 transmits the image of where the user is aiming his penile member in the direction of arrow 46 to the eyes of the user in the direction of arrow 48. The user can then avoid embarrassing spills caused by not being able to see if he is aiming his urine stream into the opening 22.

Although the side walls 20 and the end wall 18 of the housing 12 may be defined by any acceptable shape, the embodiment of FIGS. 1-2 show that a portion of the side walls 20 or the end wall 18 may be flat so that the portable urinal 10 can be stored on a horizontal surface such as a rack or table after use. In the hospital bed case, a patient may also slide the handle member 26 over a bed rail to hang the portable urinal 10 on the side of the bed.

A second embodiment of a portable urinal 10 is shown in FIG. 4. This portable urinal 10 has all the same components as the one in FIGS. 1-2, but one difference is that the projecting member 54 on the shoulder 34 of the lid member 28 contains no legs 40 or aperture 42. This projecting member 54 is permanently coupled to the handle member 26 as shown in FIG. 4, but the projecting member 54 could alternatively be permanently coupled to the housing 12. The permanent coupling can be accomplished using any kind of appropriate adhesive material or a living hinge. This permanent coupling allows for a similar kind of pivotal movement of the lid member 26 as in the first embodiment.

The housing 12, lid member 28, and handle member 26 may be made out of any suitable material, but a preferred material would be a translucent or clear plastic such as polyethylene. The portable urinal 10 can be disposed after a single use, or it can be designed to be reusable. If reuse is desired, the plastic material is ideal because it can withstand the high temperatures required for sterilization, typically around 130 degrees Celsius. The portable urinal 10 can be manufactured by any appropriate process, including blow molding or injection molding.

A third embodiment of the invention is depicted in FIG. 4 in phantom. All of the components of the first embodiment in FIGS. 1-2 are present in the third embodiment with the exception of the lid member 28. In this embodiment a mirror 36 is attached to a projection 56 extending from the handle member 26 or the housing 12 proximate to the opening 22. Any kind of permanent or removable coupling structure can be used to attach the projection to the handle member 26 or the housing 12, and this will allow portable urinals 10 with no lids to gain the novel and advantageous features of this invention.

The portable urinal 10 of this invention is economically manufacturable, is lightweight, and is easy to clean or sterilize. The portable urinal 10 may also be configured for female users as well as male users. This invention is not to be limited by what has been particularly shown and described, except as indicated by the following claims.

What is claimed is:

1. A portable urinal, comprising:
a housing defining an interior surface of the housing; and
a mirror coupled to the housing and adapted to assist a user aim urine properly into the housing when the user cannot directly see the opening in the housing.

2. The portable urinal of claim 1 further comprising:
a neck portion adjacent the opening and angled relative to an axis of the housing.

3. The portable urinal of claim 1 further comprising:
an end wall opposite the opening, wherein at least a portion of the end wall is flat for supporting the portable urinal on a horizontal surface.

4. The portable urinal of claim 1, further comprising:
a handle member coupled to the housing, the handle member being located proximate to the opening and adapted to facilitate movement and positioning of the portable urinal.

5. The portable urinal of claim 1 further comprising:
a lid member coupled to the housing and adapted to move over the opening to close the opening and prevent leaks and spills from the housing.

6. The portable urinal of claim 5, wherein the lid member further comprises:
an exterior surface;
an interior surface which completes the interior surface of the housing when the lid member covers the opening;
a shoulder adapted to removably cover the opening; and
a projecting member extending from the shoulder for coupling the lid member to the housing.

7. The portable urinal of claim 6, wherein the projecting member of the lid member is permanently connected to the housing.

8. The portable urinal of claim 1, wherein the housing and lid member are made from a plastic material.

9. The portable urinal of claim 1, wherein the mirror is created from an acrylic material.

10. The portable urinal of claim 1 wherein an orientation of the mirror is adjustable for proper viewing of the opening.

11. A portable urinal, comprising:
a housing defining an interior surface of the housing and including an end wall, at least one side wall, and an opening opposite from the end wall;
a projection extending from the housing proximate the opening; and
a mirror attached to the projection and adapted to assist a user aim urine properly into the housing when the user cannot directly see the opening in the housing.

12. The portable urinal of claim 11 further comprising:
a neck portion adjacent the opening and angled relative to an axis of the housing.

13. The portable urinal of claim 11, wherein at least a portion of the end wall is flat for supporting the portable urinal on a horizontal surface.

14. The portable urinal of claim 11, further comprising:
a handle member being coupled to the housing, the handle member being located proximate to the opening and adapted to facilitate movement and positioning of the portable urinal while in use or in storage.

15. The portable urinal of claim 11, wherein the mirror is created from an acrylic material.

16. The portable urinal of claim 14, wherein the projection is attached to the housing at the base of the handle member so that the user can move the mirror and hold onto the handle member with one hand.

17. The portable urinal of claim 11 further comprising:
a lid member coupled to the housing to selectively close and seal the opening, the mirror being mounted on the lid member.

18. The portable urinal of claim 11 wherein the projection allows for a variety of orientations of the mirror relative to the opening.

19. A method of using a portable urinal, the portable urinal including a housing with an angled neck portion where an opening in the housing is located, a lid member or projecting member coupled to an exterior surface of the housing proximate the opening, and a mirror attached to the lid member or projecting member, the method comprising:
placing the portable urinal in an approximately horizontal position with the opening facing toward a user's penile member;
orienting the mirror so that the user can see where he is aiming his penile member; and
urinating through the opening and into the portable urinal.

20. The method of claim 19 further comprising:
storing the portable urinal in an upright position with the opening at the top after urination.
